(19) United States
(12) Patent Application Publication
(10) Pub. No.: US 2017/0128663 A1
(43) Pub. Date: May 11, 2017

(54) IV Set and IV Set System Management

(71) Applicant: Somnus Medical, LLC, Pleasant Grove, UT (US)

(72) Inventors: Lucas Reichert, Roosevelt, UT (US); Edwin T. Bulloch, Vernal, UT (US); Anthony Clark Harward, Provo, UT (US)

(73) Assignee: Somnus Medical, LLC

(21) Appl. No.: 14/934,030

(22) Filed: Nov. 5, 2015

(51) Int. Cl. A61M 5/14

(52) U.S. Cl. CPC A61M 5/1418

(57) ABSTRACT

An IV set and IV set system management device and associated systems and methods. The IV set and IV set system management device can include a body portion defining an opening to receive an IV set therein to couple the IV set and IV set system management device to the IV set. The IV set and IV set system management device can also include a plurality of protrusions extending from the body portion. In addition, the IV set and IV set system management device can include one or more recesses defined, at least in part, by at least one of the protrusions. The protrusions of the IV set and IV set system management device can be configured to mate with the one or more recesses of the IV set and IV set system management device, thus facilitating coupling of the IV set and IV set system management device with a similarly configured second IV set and IV set system management device.

### BACKGROUND

One of the major benchmarks of medical care was the introduction of an Intravenous (IV) set to access the circulatory system of a patient, enabling the administration of fluids and medications in a controlled, predictable manner. The typical IV set includes a primary fluid flow line of tubular construction with one or more access points. Some of these access points can comprise access ports that allow the administration of medications through either a syringe by push or by infusion through another IV set (primary or secondary). A primary access point is located at one end of the IV set with a fluid source, such as normal saline or some other carrier fluid. A spike and drip chamber assembly is positioned at a terminus or proximal end of the flow line with means for attachment to a patient injection site on a distal end. Secondary flow lines may be combined with the primary flow line with similar construction options.

Use of IV sets has now become ubiquitous at every stage of medical care, from the site of an accident or injury, through transport to the hospital, during emergency room and surgical procedures and potentially continuing into the ICU and general hospital care. At each successive stage of procedure or treatment, different medical personnel typically become involved. Normally, later stage medical personnel will not have actual firsthand experience with a patient and the various multiple attached IV sets. Often, they may have to guess as to the purpose and procedure associated with each previous IV set, including what medications may have been administered. Usually, some form of record or identification is written or provided with respect to each IV set, the applied medications, and various details regarding patient care. Usually this is in the form of a medical chart that accompanies the patient, providing a history of such procedures. Unfortunately, there may be confusion as to the specific procedures applied with respect to an IV set based upon lack of proper interpretation or inaccuracy of description.

When multiple medical personnel are involved, the combinations of procedures involving the multiple IV sets become even more complex. This is particularly true with respect to surgical procedures in an operating room. In addition to the surgical staff having to focus on specific medical problems, other personnel may be involved, such as an anesthesia provider who typically administers anesthesia medications via the IV set. From an anesthesia provider's view, one primary concern can involve determining which IV set has the carrier fluid that he/she can use to push any needed medication (i.e., which IV set, including spike/drip chamber, goes directly to the patient).

In the operating room, or during transport to and from the operating room, the anesthesia provider must be prepared and able to quickly modify medication levels as required by hemodynamic changes in the patient's state of being, as well as anesthesia demands and unexpected emergencies. Confusion as to the correct identity of a given IV set can be a serious risk to the patient and a great frustration to attending medical personnel. The general absence of standardized labels, standardized positioning requirements along the IV sets, and inadequate IV port or access identification present an ongoing medical risk.

In addition, there are inherently many basic problems associated with the general use of current IV sets in any given situation. For example, constant concerns exist with respect to the proper introduction of a medication at an access point, the rate of fluid delivery through the flow line, the adequacy of mixture of medications within the IV set(s), the delay in transit time of a particular medication to the injection site, the amount of fluid in the flow line ahead of a newly administered drug, and maintenance of a clean and functional injection site with the patient. When multiple IV sets are combined these various problems are magnified in complexity because of the increased amount of medications and hardware used as part of the compilation of IV sets, as well as the diverse medical personnel monitoring the same compilation of IV sets.

The participation of numerous personnel using the same compilation of IV sets often results in competition for physical access by the user to access ports, control of delivery rates with valves, making modifications in the respective IV sets, etc. Maintaining convenient access to the correct IV set and ensuring proper line identification is the responsibility of each individual attempting to utilize the IV set at the same time. This competition for immediate access by multiple users can add considerable additional stress to an already stressful environment.

These problems take on a new dimension of risk in the more dynamic environment of an operating room, where decisions must be made immediately and acted upon under conditions of great urgency. Each member of the medical team must be assured of quick and certain access to the component IV set(s) associated with his or her area of attention and responsibility. In addition to proper identification of an IV set, each individual must be capable of accessing the access points and other structural aspects of the IV set or compilation of IV sets for administering the required medication or procedure, without interfering with the activities of other medical personnel attending to the patient through the same IV set or compilation of IV sets.

To meet these needs, various temporary and rudimentary methods of identifying individual IV sets and of bundling these together to avoid entanglement have been applied. For example, individual IV sets may be tagged or labeled at a single site by the attending physician or other personnel to identify its purpose. However, there is no standardization of such practices, thus leading to confusion and wasted time spent looking up and down the IV set for identification. Other attempts at line identification have included coloring the actual IV set itself. This may be a problem, however, because medications are often colored themselves. For example, a yellow IV set containing therein a cancer medication that is blue could provide a misrepresentative or false appearance in the form of a green IV set.

To avoid entanglement, the various IV sets may be taped together or otherwise generally clipped into a bundle. This grouping of multiple IV sets with intermittent access ports, however, may become very confusing to multiple users of the compilation of IV sets, and is also very time consuming. Access points allow the various attending personnel to administer medications through a syringe by push or by infusion through an additional IV set. Not only does this additional structure add to the complexity of the compilation of IV sets with respect to possible line entanglement, but it also limits access for the practitioner using a more proximal access port. For example, when a fluid restriction is being observed (e.g., pediatrics, kidney failure, or complex surgery case), pushing a medication through an access point far from the patient becomes a problem. Boluses of fluid must be administered to deliver the medication from the proximal access points to a distal end terminating at the patient.

In view of these ongoing problems, medical personnel continue to make ad hoc adaptations in an effort to mitigate the confusion associated with current IV set practices. Despite these various solutions, current use of IV sets still requires considerable patience and a high degree of attention on the part of medical personnel to deal with the ongoing difficulties associated with such current IV sets.

### BRIEF DESCRIPTION OF THE DRAWINGS

Features and advantages of the invention will be apparent from the detailed description which follows, taken in conjunction with the accompanying drawings, which together illustrate, by way of example, features of the invention; and, wherein:

FIG. 1 is an illustration of an IV set and IV set system management device coupled to an IV set in accordance with an example of the present disclosure.

FIG. 2 shows the IV set and IV set system management device of FIG. 1 in isolation.

FIG. 3 is an illustration of an IV set and IV set system management device coupled to an IV set in accordance with another example of the present disclosure.

FIG. 4 shows the IV set and IV set system management device of FIG. 3 in isolation.

FIG. 5 is a cross-sectional view of an IV set and IV set system management device in accordance with an example of the present disclosure.

FIGS. 6A and 6B illustrate an IV set management system, wherein two IV set and IV set system management devices are coupled together, in accordance with an example of the present disclosure.

FIG. 7 is a perspective view of the IV set management system of FIG. 6B.

FIGS. 8A-8C illustrate different end views showing different positions of the IV set management system of FIG. 6B.

FIG. 9 illustrates an IV set management system, wherein three IV set and IV set system management devices are coupled together, in accordance with an example of the present disclosure.

FIG. 10 is an end view of the IV set management system of FIG. 9.

FIG. 11 is an illustration of an IV set and IV set system management device for coupling to an IV set in accordance with an example of the present disclosure.

FIG. 12 shows the IV set and IV set system management device of FIG. 11 coupled to an IV set.

FIG. 13 illustrates an end view of an IV set management system, wherein four IV set and IV set system management devices are coupled together, in accordance with an example of the present disclosure.

FIG. 14 is a perspective view of an IV set and IV set system management device in accordance with another example of the present disclosure.

FIG. 15 is a perspective view of an IV set and IV set system management device in accordance with another example of the present disclosure.

FIG. 16 is a cross-sectional view of an IV set and IV set system management device in accordance with another example of the present disclosure.

FIG. 17 is a perspective view of an IV set and IV set system management device in accordance with another example of the present disclosure.

FIG. 18 is an illustration of a protrusion of an IV set and IV set system management device having a tip configured as a coupling enhancement feature, in accordance with an example of the present disclosure.

FIGS. 19A and 19B illustrate how an IV set and IV set system management device having rod protrusions can couple with another similarly configured IV set and IV set system management device, in accordance with an example of the present disclosure.

FIGS. 20A and 20B illustrate how an IV set and IV set system management device having rod protrusions can couple with another similarly configured IV set and IV set system management device, in accordance with another example of the present disclosure.

FIGS. 21A-21D illustrate how an IV set and IV set system management device having rod protrusions can couple with another similarly configured IV set and IV set system management device, in accordance with several examples of the present disclosure.

FIG. 22 is a cross-sectional view of an IV set and IV set system management device in accordance with another example of the present disclosure.

FIG. 23 is a cross-sectional view of an IV set and IV set system management device in accordance with yet another example of the present disclosure.

FIG. 24A is a cross-sectional view of protrusions of an IV set and IV set system management device having coupling enhancement features, in accordance with an example of the present disclosure.

FIG. 24B is a cross-sectional view of the protrusions of FIG. 24A engaged with similarly configured protrusions.

FIG. 25A is a cross-sectional view of protrusions of an IV set and IV set system management device having coupling enhancement features, in accordance with another example of the present disclosure.

FIG. 25B is a cross-sectional view of the protrusions of FIG. 25A engaged with similarly configured protrusions.

Reference will now be made to the exemplary embodiments illustrated, and specific language will be used herein to describe the same. It will nevertheless be understood that no limitation of the scope of the invention is thereby intended.

### DETAILED DESCRIPTION

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

An initial overview of technology embodiments is provided below and then specific technology embodiments are described in further detail later. This initial summary is intended to aid readers in understanding the technology more quickly but is not intended to identify key features or essential features of the technology nor is it intended to limit the scope of the claimed subject matter.

At the outset, an IV set is intended to mean a single IV line. An IV set system is intended to mean at least a plurality of IV sets within the IV set system. The IV set system can comprise additional elements operable within the IV set, such as a merging fluid pathway, a manifold, various access points, etc. An access point is intended to mean any point along the IV set in which access is provided to the fluid flow within the IV set. An access point can include such elements as access ports, spike/drip chambers, patient interconnect structures, fluid interconnect means, etc. An access port is intended to mean a specific type of access point that facilitates access to the fluid flow within the IV set, such as by push (e.g., using a syringe) or by infusion (e.g., through fluid coupling of another IV set). Although the IV set and IV set system management devices disclosed herein are discussed in the context of managing and organizing IV sets, it should be recognized that the IV set and IV set system management devices can be utilized to manage and organize any suitable line, cable, etc. (e.g., medical lines, communication lines, data lines, optical cables, power cables, etc.).

Historically, the use of the IV set has been segmented within various progressive stages of the patient experience. For example, at the scene of an accident, paramedics may apply an IV set for temporary relief and application of emergency procedures. Upon arrival at the emergency room, a new IV set may be substituted upon identification of particular needs of the patient. Once the patient is stabilized and an initial diagnosis is made, changes may again be required in the IV set or the now compilation of IV sets. Eventually, the patient may be moved to a hospital ward for further treatment and evaluation. Once again, further modifications may be needed with respect to the patient's IV. If surgery is ultimately required, the patient is typically prepared, which preparations can include additional modifications in IV set up as needed for delivery of anesthesia and other required medications. Finally, upon completion of the surgery, the compilation of IV sets may again be adapted for normal hospital usage. As such, the reality of changing patient environments and medical needs, coupled with the recognized problems associated with current IV set technology, calls for a dynamic IV set system and solution that can be adapted to meet the numerous requirements of secure and reduced risk administration of IV medications, as well as improved convenience of use for medical personnel.

Accordingly, an IV set and IV set system management device (hereinafter IV set and IV set system management device, IV set management device or management device) and associated methods of use are disclosed that facilitates control, management, and organization of one or more IV sets during use, avoiding entanglement issues. The IV set and IV set system management device can enable an IV set to be easily joined with other IV sets to build IV set systems, which can be subsequently separated and rebuilt by end-users in other locations within a hospital, as desired. The IV set and IV set system management device can include a body portion defining an opening to receive an IV set therein to couple the IV set management device to the IV set. The IV set management device can also include a plurality of protrusions extending from the body portion. In addition, the IV set management device can include at least one recess defined, at least in part, by at least one of the plurality of protrusions. The protrusions of the IV set management device can be configured to mate with the at least one recess of the IV set management device, thus facilitating coupling of the IV set management device with a similarly configured second IV set and IV set system management device.

In one aspect, an IV set and IV set system management device can include a body portion defining an opening to receive an IV set therein to couple the IV set management device to the IV set. The opening can have a longitudinal axis. The IV set management device can also include a plurality of fins extending from the body portion. In addition, the IV set management device can include at least one recess defined, at least in part, by at least one of the plurality of fins. The plurality of fins can be spaced apart from one another by the at least one recess along the longitudinal axis. The protrusions of the IV set management device can be configured to mate with the at least one recess of the IV set management device, thus facilitating coupling of the IV set management device with a similarly configured second IV set and IV set system management device.

In another aspect, an IV set and IV set system management device can include a body portion defining an opening to receive an IV set therein to couple the IV set management device to the IV set. The IV set management device can also include a plurality of rods extending from the body portion. In addition, the IV set management device can include at least one recess defined, at least in part, by at least one of the plurality of rods. The rods of the IV set management device can be configured to mate with the at least one recess of the IV set management device, thus facilitating coupling of the IV set management device with a similarly configured second IV set and IV set system management device.

In one aspect, an IV set management system can include a first IV set management device and a second IV set management device. Each IV set management device can have a body portion defining an opening to receive an IV set therein to couple the IV set management device to the IV set. Each IV set management device can also have a plurality of protrusions extending from the body portion. In addition, each IV set management device can include at least one recess defined, at least in part, by at least one of the plurality of protrusions. The protrusions of the first IV set management device can be configured to mate with the at least one recess of the second IV set management device, thus facilitating coupling of the first IV set management device with the second IV set management device.

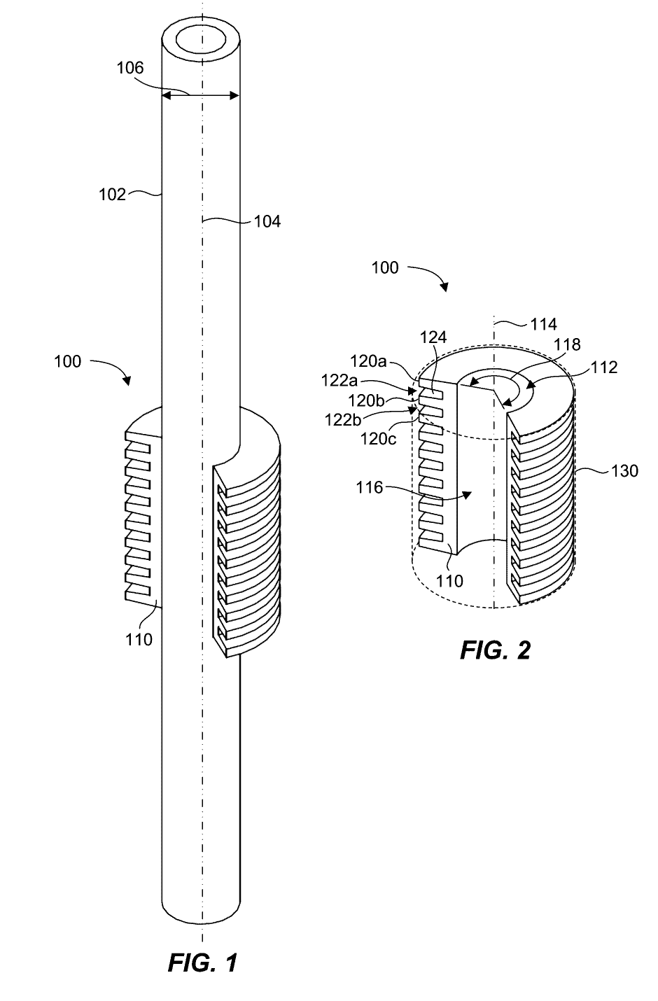

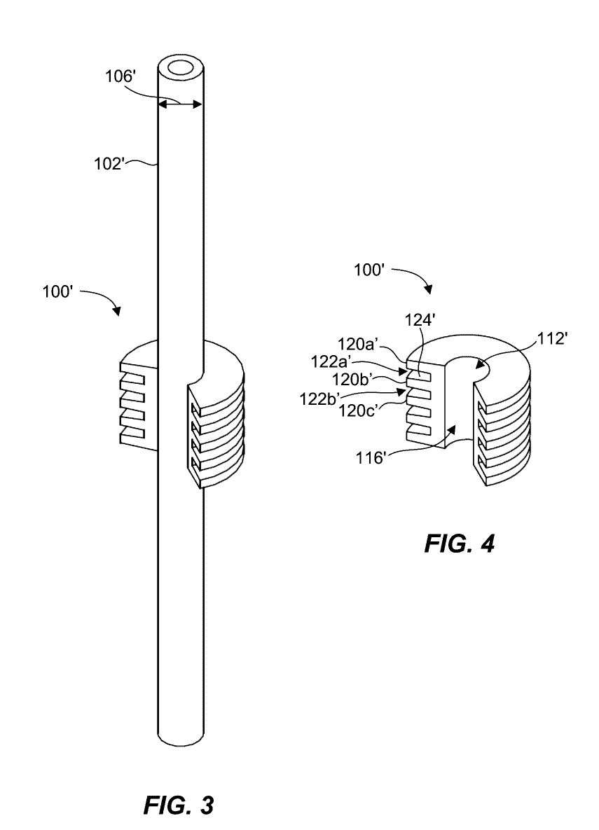

One example of an IV set management device 100 is illustrated in FIGS. 1 and 2. FIG. 1 shows the IV set management device 100 coupled to an IV set 102 and FIG. 2 shows the IV set management device 100 in isolation. The IV set management device 100 can comprise a body portion 110, which can define an opening 112 to receive the IV set 102 therein to couple the IV set management device to the IV set. The opening 112 can have a longitudinal axis 114, which can be configured to align with or be parallel to a longitudinal axis 104 of the IV set 102. The IV set management device 100 can also include a plurality of protrusions 120a-c extending from the body portion 110. For simplicity, only three protrusions are specifically identified in the figures, although more protrusions are illustrated. In addition, the IV set management device 100 can include one or more recesses 122a-b defined or formed, at least in part, by at least one of the protrusions. For simplicity, only two recesses are specifically identified in the figures, although more recesses are illustrated. The protrusions 120a-c of the IV set management device 100 can be configured to mate with the one or more recesses 122a-b of the IV set management device, thus facilitating coupling of the IV set management device 100 with a similarly configured second IV set management device (e.g., IV set management device 100′ of FIGS. 3 and 4).

The protrusions 120a-c and the one or more recesses 122a-b of the IV set management device 100 can define a mating or coupling interface 124, such that the IV set management device is configured to mate with the second IV set management device having a similarly configured mating or coupling interface (e.g., mating or coupling interface 124′ of the IV set management device 100′ of FIGS. 3 and 4). In one aspect, the protrusions of one IV set management device can intermingle or mesh with the protrusions of another IV set management device. For example, the protrusions 120a-c of the IV set management device 100 can intermingle or mesh with the protrusions 120a′-c′ of the IV set management device 100′ by the protrusions 120a-c of the IV set management device 100 being received by recesses 122a′-b′ between the protrusions 120a′-c′ of the IV set management device 100′. Similarly, the protrusions 120a′-c′ of the IV set management device 100′ can intermingle or mesh with the protrusions 120a-c of the IV set management device 100 by the protrusions 120a′-c′ of the IV set management device 100′ being received by recesses 122a-b between the protrusions 120a-c of the IV set management device 100.

In one aspect, the protrusions of one IV set management device can all be similarly configured (e.g., in shape, size, orientation, etc.), such that any of the protrusions can mate with any one of the configured recesses defined by similarly configured protrusions of another IV set management device. Although all of the protrusions can be similarly configured in shape, size, orientation, etc., it should be recognized that the protrusions of an IV set management device can differ in at least one aspect or characteristic and can still mate with a recess of another IV set management device. For example, not all protrusions of an IV set management device need be the same length or extend the same distance from the body portion. Such a configuration can still be effective to define one or more recesses that are configured to mate with the protrusions of various lengths.

In one aspect, protrusions can be configured as “fins” as shown in FIGS. 1-4. Such fin protrusions 120a-c can extend annularly from the body portion 110. The fin protrusions 120a-c are illustrated as being oriented perpendicular to the longitudinal axis 114 of the opening 112, although protrusions or fins can be oriented at any suitable angle relative to a longitudinal axis of an opening. In another aspect, the protrusions can be spaced apart from one another by the recesses along a longitudinal axis of an opening. For example, the fin protrusions 120a-c can be spaced apart from one another by the recesses (e.g., separated by a series of gaps) along the longitudinal axis 114 of the opening 112. Typically, recesses and protrusions are the same size but, in some embodiments, recesses and protrusions can be different in size.

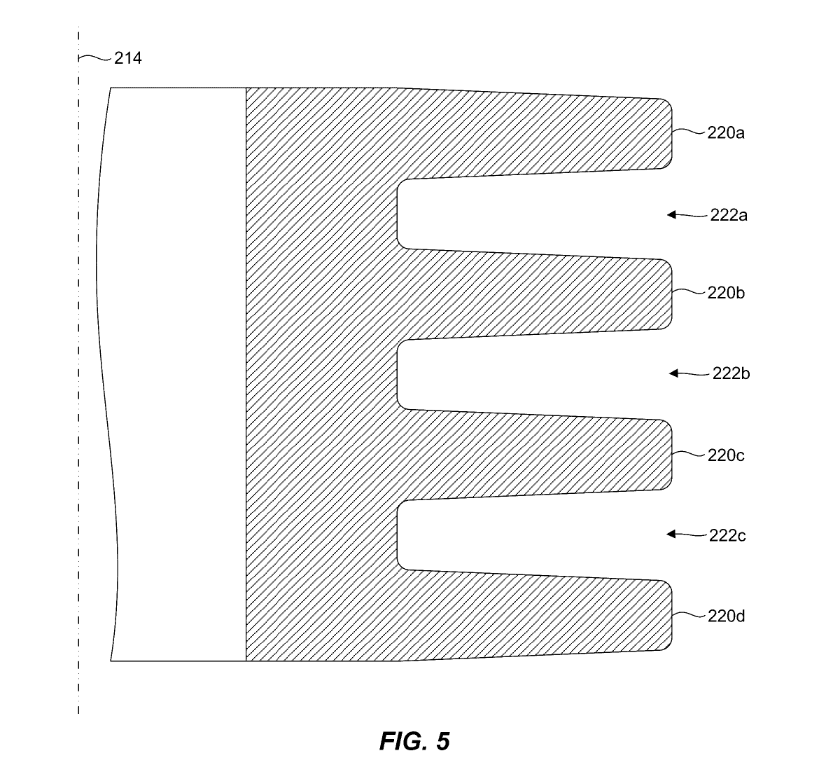

In one aspect, shown in FIG. 5, one or more recesses 222a-c of an IV set management device can comprise an inverted shape of the protrusions 220a-d of the IV set management device. For example, the recess 222a is an inverted shape (e.g., an inverted cross-sectional shape with respect to the longitudinal axis 214) of the protrusion 220b. As illustrated, the protrusions 220a-d can be tapered to facilitate meshing or intermingling with other protrusions for coupling IV set management devices to one another. Protrusions and recesses may be formed with chamfers or rounds on edges or corners as is common manufacturing practice. Such chamfers or rounds do not detract from the overall shape of the protrusions or recesses. Thus, a protrusion that has a chamfer on a distal edge and a recess that has a round on a proximal corner can still be deemed to be inverted shapes of one another. In other words, minor local variations in form between a protrusion and a recess due to manufacturing practices and/or variations of form within manufacturing tolerances are deemed insignificant when considering whether the protrusion and the recess are inverted shapes of one another.

With further reference to FIGS. 1 and 2, the IV set management device 100 can include any suitable number of protrusions 120a-c and recesses 122a-b, which can be of any suitable size or configuration. As shown in the figures, the IV set management device 100 can include a plurality of protrusions 120a-c that defines a plurality of recesses 122a-b. In one embodiment, an IV set management device may include only two fin protrusions that define only a single recess. An IV set management device can include more than two protrusions that define only a single recess, such as when the protrusions are configured as “rods,” as described below.

In one aspect, an outer boundary 130 of the protrusions 120a-c can be defined at least in part by any suitable three-dimensional shape, such as a cylinder as shown in the figures, a cone, a pyramid, a cuboid, etc. A cylindrical-shaped outer boundary can facilitate variable attachment angles (i.e., as measured about the longitudinal axis 114 of the opening 112) for coupling with another IV set management device, as described further below.

The body portion 110 can define a lateral open end 116 that provides access to the opening 112 for the IV set 102 to facilitate insertion or removal of the IV set during assembly or use. In one aspect, the body portion 110 can extend to an angle 118 greater than 180 degrees about the longitudinal axis 114 of the opening 112 to define the lateral open end 116, such that the IV set 102 is laterally received in the opening 112 and secured therein by the body portion 110. Thus configured, the lateral open end 116 is sized smaller than an outer diameter 106 of the IV set 102. As a result, the IV set 102 and/or the IV set management device 100 can elastically deform during attachment or removal of the IV set management device 100 to permit the IV set 102 (which can deform to a smaller outer dimension) to pass through the lateral open end 116 (which can deform to an enlarged dimension) of the IV set management device 100.

In one aspect, the openings of the IV set management devices disclosed herein can be configured to receive IV sets of any diameter, which can facilitate coupling a variety of different sized IV sets together. For example, the similarly configured IV set management devices 100, 100′ of FIGS. 2 and 4 can have openings 112, 112′ that are configured to receive IV sets 102, 102′ having different diameters 106, 106′, as shown in FIGS. 1 and 3, respectively.

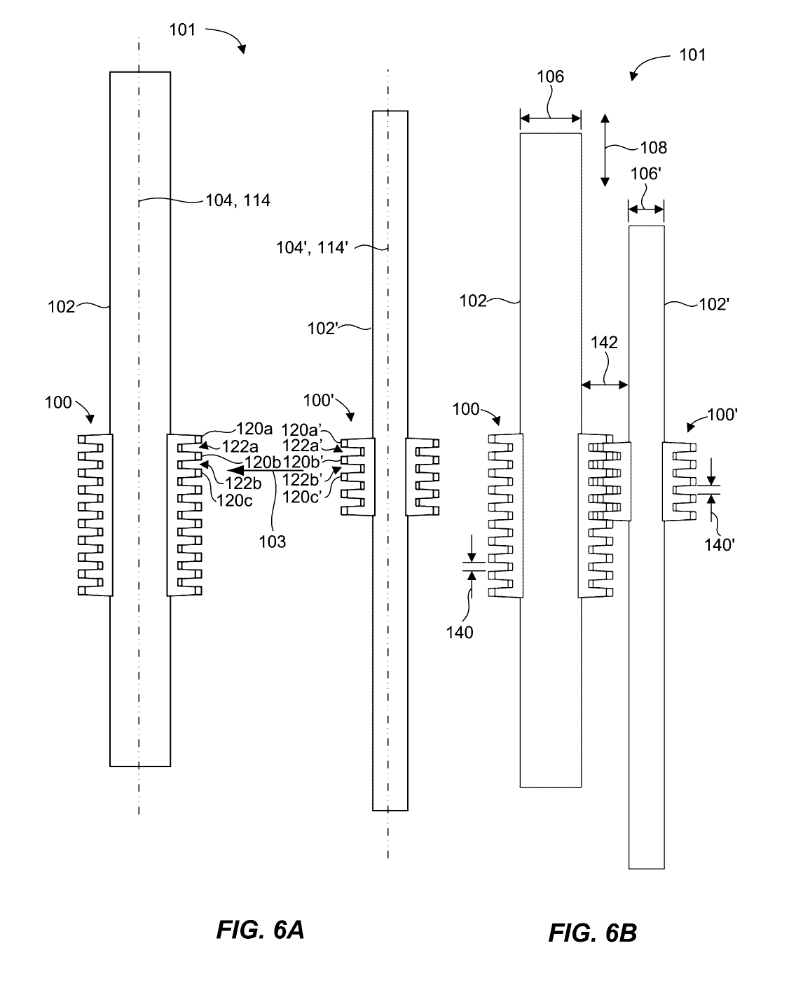

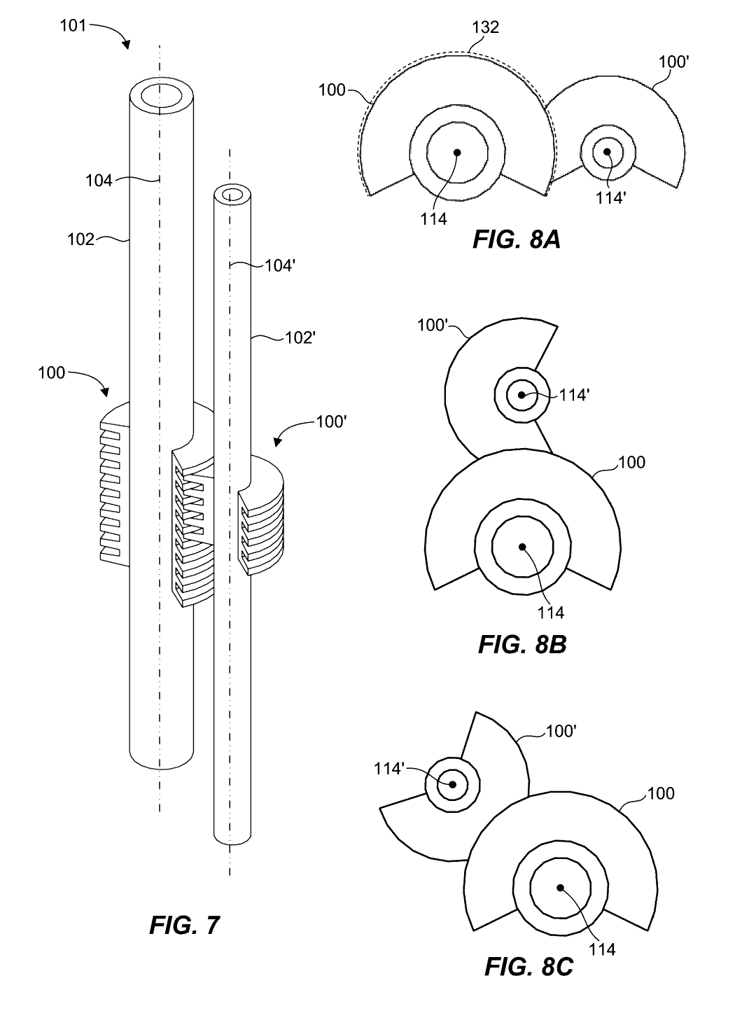

The IV set management devices 100, 100′ can be coupled to one another as shown in FIGS. 6A-8B, and can form an IV set management system 101. With particular reference to FIGS. 6A and 6B, the IV set management system 101 can include at least two IV set management devices, such as the IV set management device 100 of FIGS. 1 and 2 and the IV set management device 100′ of FIGS. 3 and 4. The IV set management device 100 may be associated with a primary IV set (e.g., IV set 102) and the IV set management device 100′ may be associated with a secondary IV set (e.g., IV set 102′). The protrusions 120a-c of the IV set management device 100 are configured to mate with the recesses 122a′-b′ of the IV set management device 100′, and the protrusions 120a′-c′ of the IV set management device 100′ are configured to mate with the recesses 122a-b of the IV set management device 100, thus facilitating coupling of the IV set management device 100 with the IV set management device 100′, as shown in FIGS. 6A-7. In this case, the attachment direction 103 is generally perpendicular (e.g., radial) to the longitudinal axes of the IV sets and the openings 104, 104′, 114, 114′. The IV set management devices 100, 100′ can be pushed toward one another to engage the protrusions 120a-c, 120a′-c′ into the mating recesses 122a-b, 122a′-b′. In some cases, a slight rolling or rotation of the IV set management devices 100, 100′ about their respective longitudinal axes 104, 104′ can facilitate engagement and coupling, or disengagement or decoupling of the IV set management devices. Although the IV set management devices 100, 100′ have similarly configured mating or coupling interfaces, the IV set management devices 100, 100′ have different numbers of protrusions 120a-c, 120a′-c′ and can be of different overall size dimensions. Thus, it should be recognized that mating IV set management devices can have any suitable number of protrusions and recesses, as well as any suitable overall size dimensions.

The configuration of the protrusions and recesses of IV set management devices can facilitate a variety of coupling options for the similarly configured IV set management devices. For example, as illustrated in FIG. 6B, the multiple protrusions 120a-c, 120a′-c′ on each IV set management device can enable the IV set management devices 100, 100′ to be coupled to one another in a variety of relative positions in a longitudinal direction 108 so long as at least one protrusion is received within a recess. In addition, as illustrated in FIGS. 8A-8C, the IV set management devices 100, 100′ can have a variable attachment angle (i.e., as measured about the longitudinal axes 114, 114′ of the respective openings). The attachment angles can be varied depending on the orientation of the IV set management devices 100, 100′ about their respective longitudinal axes 114, 114′, thus leading to the variations of relative position of the IV set management devices 100, 100′ in FIGS. 8A-8C. The attachment angles can be continuous variable within the radial angular range of available attachment. In one aspect, the angular range of available attachment can be greater than 180 degrees, which can coincide with the extension of the body portions 110, 110′ about the longitudinal axes 114, 114′ of the opening to define the lateral open ends 116, 116′. Having a range of angular positions available for attachment can contribute to a “multi-directional” coupling attribute of the IV set management devices 100, 100′ in that, unlike typical male/female couplings, there is no one specific direction in which the IV set management devices 100, 100′ must be positioned or oriented relative to one another in order to couple with one another. Thus, an IV set management device associated with a “secondary” IV set can be attached to an IV set management device associated with a “primary” IV set in any position around the outer boundary (e.g., circumferential surface) of the primary IV set IV set management device. As disclosed herein, any number of secondary IV set IV set management devices can be attached to the primary IV set IV set management device at any available location.

In one aspect, an outer boundary (as discussed above) or a projected outer boundary of the protrusions of an IV set management device can facilitate variable attachment angles for coupling IV set management devices to one another. A projected outer boundary 132 of the protrusions of the IV set management device 100 is shown as an example in FIG. 8A. The projected outer boundary 132 of the protrusions can be in a direction parallel to the longitudinal axis 114 of the opening. An outer boundary or a projected outer boundary having some curvature or multiple lateral sides (such as a cuboid) can provide multiple attachment angles. A projected outer boundary of the protrusions can comprise any suitable shape, which can include, in whole or in part, a circular shape (e.g., the semicircular shape as in FIG. 8A), an elliptical shape, a rectangular shape, a triangular shape, a polygonal shape, and/or a free-form shape.

In one aspect, spacing between adjacent coupled IV sets can be less than a diameter of one of the IV sets. For example, the spacing distance 142 between the IV set management devices 100, 100′ is less than the diameter 106 of the IV set management device 100. This facilitates tight clustering of IV sets and IV set systems, while still maintaining organization across the various IV sets.

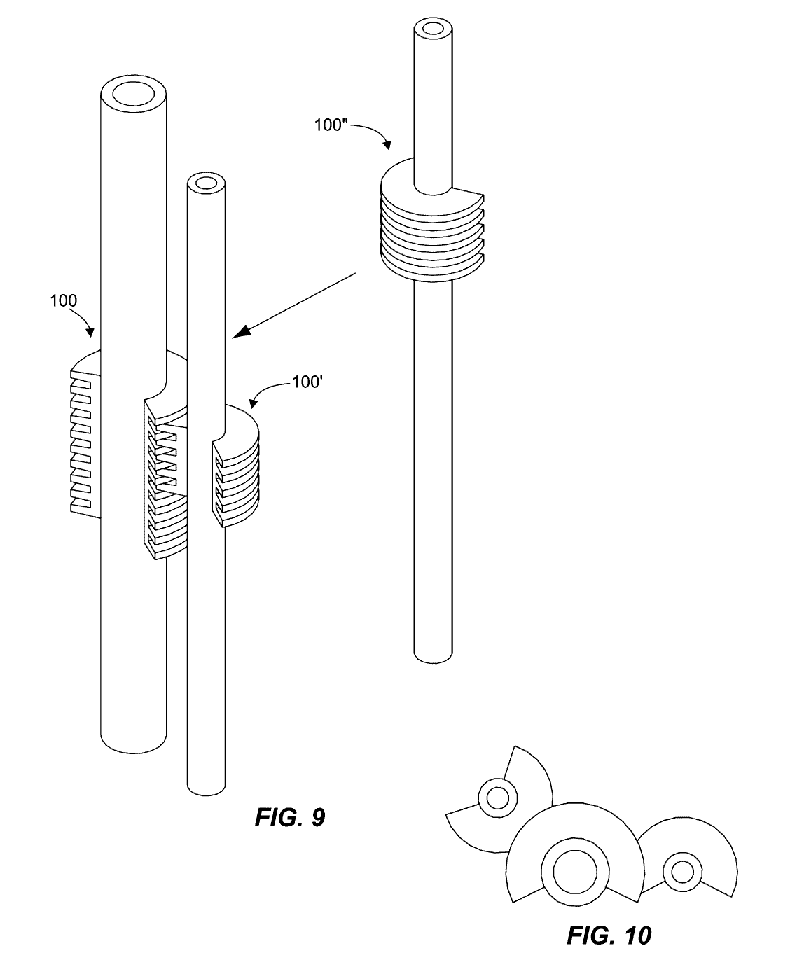

FIGS. 9 and 10 illustrate the coupling of a third IV set management device 100″ to the IV set management devices 100, 100′ that are already coupled to one another. The third IV set management device 100″ has mating or coupling interfaces that are similarly configured to the mating or coupling interfaces of the IV set management devices 100, 100′, thus facilitating coupling of the third IV set management device 100″ with one or both of the IV set management devices 100, 100′. For example, the protrusions of the third IV set management device 100″ can be configured to mate with the recesses of the IV set management devices 100, 100′. In other words, each one of the IV set management devices 100, 100′, 100″ can be directly coupleable to the other two IV set management devices, individually or in combination. In one aspect, multiple IV set management devices can be coupled together in a series arrangement or configuration to “chain” multiple IV sets together.

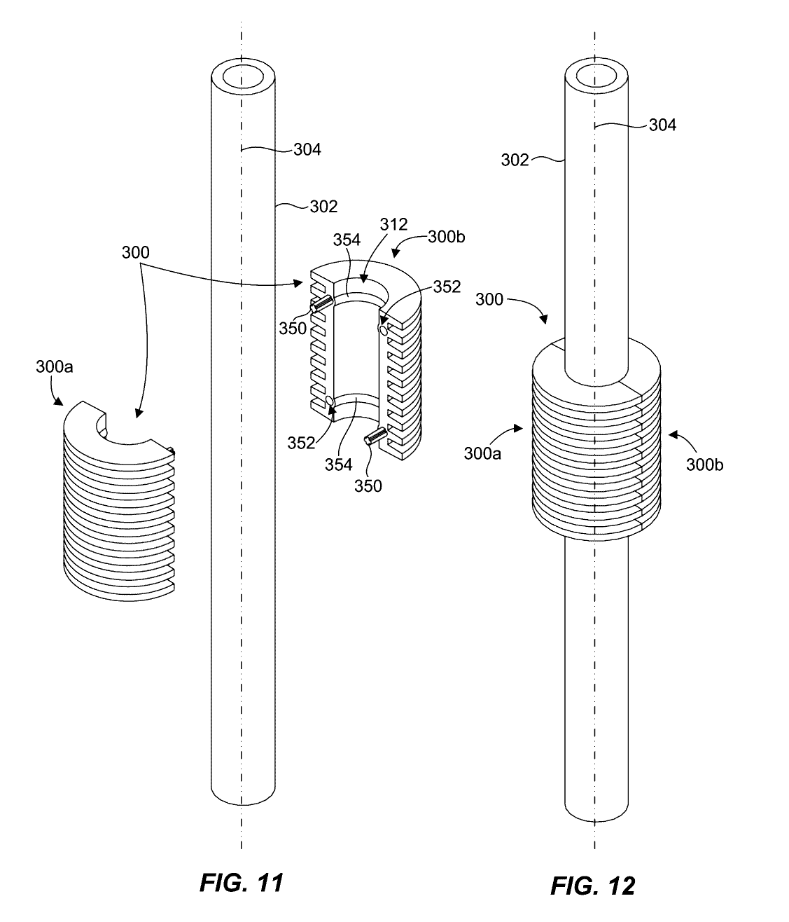

FIGS. 11 and 12 illustrate an IV set management device 300 in accordance with another example of the present disclosure. The IV set management device 300 is similar in many respects to the IV set management devices 100, 100′, 100″ discussed above. In this case, instead of having a lateral open end to facilitate coupling to an IV set, the IV set management device 300 includes a full 360 degree structure having multiple portions 300a, 300b that are coupleable to one another about an IV set 302. The IV set management device portions 300a, 300b can be two physically distinct portions, as shown in the figures, or the IV set management device portions 300a, 300b can be coupled to one another via a hinge in a “clamshell” configuration (not shown). The IV set management device portions 300a, 300b can include coupling features, such as tabs 350 and mating receptacles 352, to secure the IV set management device 300 about the IV set 302. The coupling features that secure the IV set management device 300 about the IV set 302 can be configured for a permanent coupling or a removable coupling. It should be recognized that a single, unitary IV set management device can be configured in a similar manner as the IV set management device 300 having multiple portions, in that an opening for an IV set can exist without a lateral open end to facilitate coupling to the IV set.

In one aspect, at least one of the IV set management device portions 300a, 300b can define a friction enhancing feature 354 in the opening 312 to engage the IV set 302 and maintain the IV set management device 300 in a fixed position relative to the IV set 302 during use. For example, the IV set management device portions 300a, 300b can comprise one or more protuberances, as illustrated, extending into the opening 312 to resist relative movement between the IV set management device 300 and the IV set 302 in a direction parallel to a longitudinal axis 304 of the IV set 302. In another aspect, the IV set management device 300 can be fixed (e.g., glued) to the IV set 302 to prevent relative movement between the IV set management device 300 and the IV set 302. Those skilled in the art will recognize other ways to fix the IV set management device 300 about the IV set 302.

Alternatively, the IV set management device 300 can be configured to slide along the IV set 302 for repositioning and then be held in place. In one aspect, the friction enhancing feature 354 can be configured to facilitate both a clamping feature about the IV set and a sliding feature, wherein a sliding force can be applied to the IV set management device 300 to move it to a different location along the length of the IV set 302. The clamping force applied by the friction enhancing feature 354 in this case will be less than if the IV set management device 300 were intended to be fixed in place as in the example discussed above, but still great enough to hold it in place about the IV set 302 once the sliding force is removed. Another way slidability can be accomplished is by configuring the IV set management device 300 to comprise a certain diameter, such that an interference coupling is created with respect to the IV set 302. In this case, the IV set management device 300 can couple to the IV set 302 in a manner such that a slight clamping force is applied to the IV set 302 to hold it in any one position, and wherein the clamping force still facilitates sliding of the IV set management device 300 along the IV set 302 upon a suitable force being applied to the IV set management device 300 (e.g., a force in a direction along the IV set 302). Thus, IV set management devices associated with primary or secondary IV sets may be fixed in their corresponding positions or may be slidably attached to the IV sets in approximate corresponding positions. Those skilled in the art will recognize that these features can be applicable to any embodiment. In one aspect, multiple IV set management devices can be connected to a single IV set and can be longitudinally spaced apart from one another by any suitable spacing, such as from about 6 to 8 inches apart along the IV set, which can facilitate coupling multiple IV sets together along any suitable length of the IV sets.

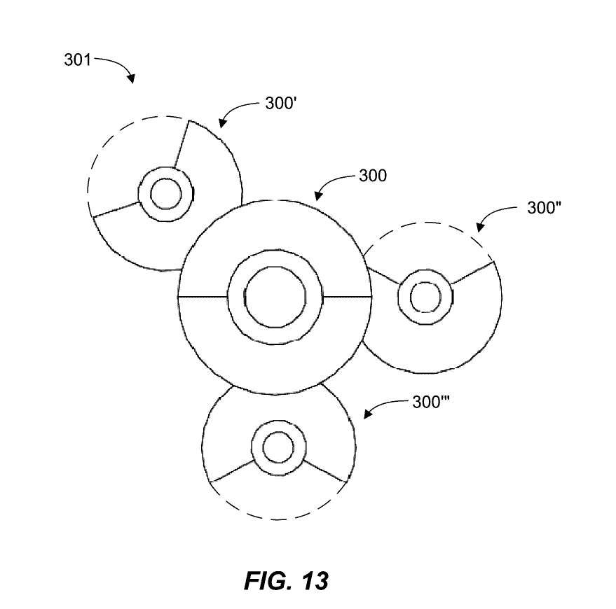

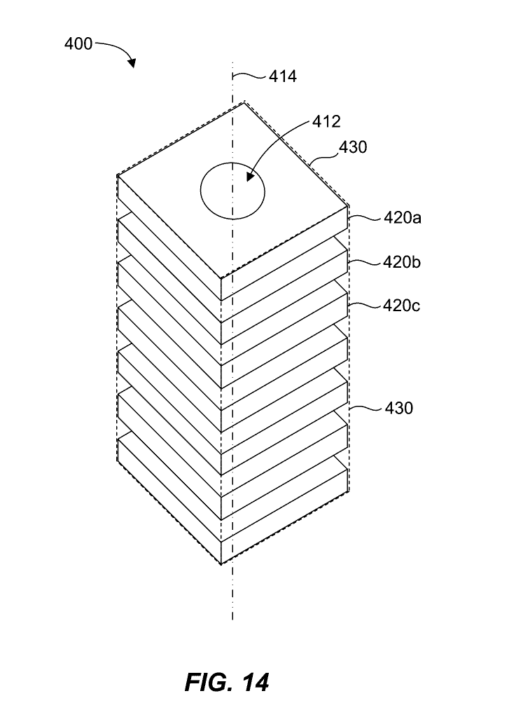

FIG. 13 illustrates an IV set management system 301 in accordance with another example of the present disclosure. The system 301 can include the IV set management device 300 of FIGS. 11 and 12, which may be associated with a primary IV set, and any suitable number of additional IV set management devices 300′, 300″, 300′″, which may be associated with secondary IV sets. Due to the lack of a lateral open end in the IV set management device 300, the secondary IV set IV set management devices 300′, 300″, 300′″ can be attached to the IV set management device 300 anywhere around a 360 degree angular range of attachment. The secondary IV set IV set management devices 300′, 300″, 300′″ can be configured similar to the IV set management device of FIG. 4, which has a lateral open end to facilitate coupling to an IV set. Alternatively, the IV set management devices 300′, 300″ and 300′″ can be configured similar to the IV set management device of FIGS. 11 and 12 (i.e., with no lateral open end) (see, for example, the full 360 degree structures as represented by dotted lines in FIG. 13), wherein coupling between any IV set management device on any IV set (primary or secondary) can be anywhere around a 360 degree angular range of attachment.

The IV set management devices disclosed herein can facilitate the organization (e.g., maintained untangled) and management of IV set lines throughout a wide range of situations and environments, such as in the OR, ICU, or for other multiple line medical device procedures or patient care processes. Secondary IV sets may be removed from a multiple IV line system by the end-user at will. A secondary IV set and associated IV set management device may be uncoupled from the primary IV set management device, or removed from its associated IV set management device, leaving the IV set management device in place coupled to the primary IV set management device, which can then be coupled to a different secondary line, as desired, for example in the ICU after leaving the OR or vice versa. After leaving the OR, new secondary lines, with their associated IV set management devices in place, may be added to a multiple line system in the ICU by attaching the secondary IV set management devices to the mating primary IV set management devices, which may be distributed along a certain length of the IV sets. Primary and secondary IV set management devices can be positioned in the factory at corresponding points along their lengths. For convenience, a multiple line IV set can come pre-connected in the package provided to the end-user. This will save time as well as inform the end-user how the IV set management system works.

In one aspect, the IV set management devices disclosed herein can include a color and/or shape to facilitate identification of an IV set and/or to differentiate the various IV sets of an IV set system. Such identifying colors or shapes can be allocated and standardized for medications associated with blood, pain medication, anesthesia, etc., as well as any others and/or combinations of these. In one aspect, identifying indicia can form part of the original construction of the IV set management devices, being fabricated as part of the original structure. Alternatively, identifying indicia can be manufactured independent of the IV set management devices, and later applied or otherwise associated with the various IV set management devices associated with primary and/or secondary IV sets of an IV set system subsequent to their initial manufacture. Such identifying indicia may also include color, overall geometric shape, protrusion configuration, size, texture, or any other tactile and/or visual element that can enable the attending personnel to quickly and accurately identify a given IV set within the IV set system. The use of multiple or different identifying indicia (e.g., including one or more of shape, size, color, and texture) will enhance the sure identification of the various IV sets.

Because the IV set management devices associated with the various IV sets can be color coded (or otherwise uniquely identifiable by an identifying indicia), IV sets can be more easily managed during use. Marking each IV set with an IV set management device having identifying indicia enables personnel to quickly and easily identify the various flow lines of the IV set system, maintain separate alignment of these IV sets, and avoid weaving and entangling the various lines amongst each other, thus easing use of IV sets during medical situations, particularly during emergency and other stressful situations. Otherwise, without clear identification, a user might grasp what he/she believes is the correct line, subsequently realize the error, and quickly attempt to take another line and reposition it where needed. Eventually, these lines may become interwoven, further complicating the correct identification. The present invention can therefore minimize problems of line entanglement by allowing IV sets to be physically joined or coupled together in a removable manner. For example, each IV set system can comprise a primary IV set and one or more secondary IV sets joined to the primary IV set and/or an additional secondary IV set. Additionally, each IV set management device associated with each IV set can be properly coded for unique identification.

FIG. 14 illustrates an IV set management device 400 in accordance with another example of the present disclosure. In this example, protrusions 420a-c of the IV set management device 400 have an outer boundary 430 that is defined at least in part by a cuboid three-dimensional shape. This example also illustrates an outer boundary of the protrusions that is defined at least in part by a plane (e.g., a side of the cuboid shape). In addition, a projected outer boundary 430 of the protrusions 420a-c in a direction parallel to a longitudinal axis 414 of an opening 412 for an IV set is a rectangle. This configuration can facilitate variable attachment angles for coupling IV set management devices to one another. In this case, the angular range of attachment is 360 degrees about the longitudinal axis 414 of the opening 412, due to the lack of a lateral open end for coupling with an IV set, although such a lateral open end can be included. The IV set management device 400 has a mating or coupling interface that can couple with any IV set management device having a similarly configured mating or coupling interface, even if the other IV set management device has a different protrusion outer boundary, such as the generally cylindrical IV set management devices discussed above with reference to FIGS. 2, 4, and 12.

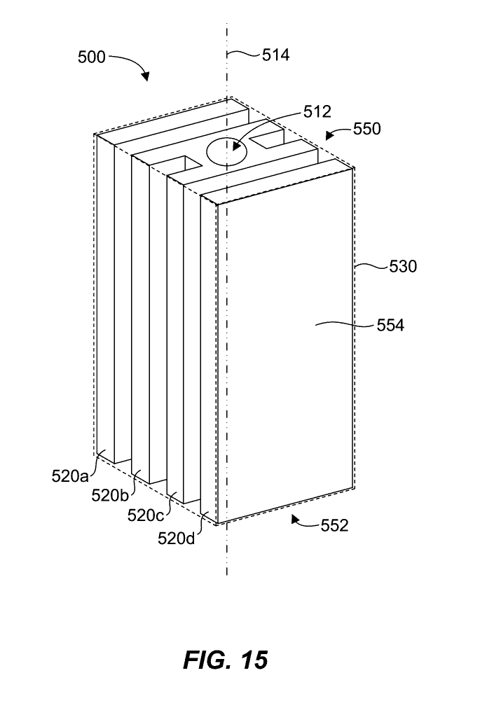

FIG. 15 illustrates an IV set management device 500 in accordance with another example of the present disclosure. This example has a protrusion outer boundary 530 that is defined at least in part by a cuboid three-dimensional shape, as with the IV set management device 400 of FIG. 14. In this case, however, protrusions 520a-d of the IV set management device 500 are oriented parallel to a longitudinal axis 514 of an opening 512 for an IV set, which is configured to be parallel to a longitudinal axis of an IV set. Thus, protrusions of an IV set management device as disclosed herein can be oriented in any suitable orientation relative to a longitudinal axis of an opening or an IV set. The orientation of protrusions 520a-d facilitates an attachment direction that is perpendicular to a longitudinal axis of an IV set. This orientation of protrusions can also expose protrusions on longitudinal ends 550, 552 of the IV set management device 500, which facilitates an attachment direction that is parallel to a longitudinal axis of an IV set. Although a lateral side 554 of the IV set management device 500 is depicted without any protrusions or recesses, it should be recognized that any side of the IV set management device 500 can include protrusions and one or more recesses for coupling with another IV set management device.

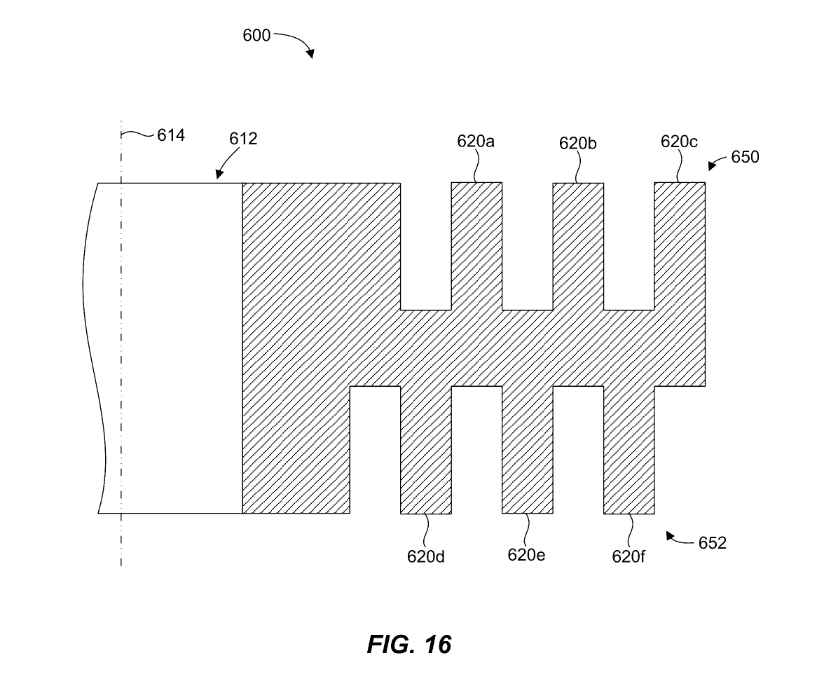

FIG. 16 illustrates a cross-section of an IV set management device 600 in accordance with another example of the present disclosure. In this example, protrusions 620a-f of the IV set management device 600 are oriented for exposure and attachment in a direction that is parallel to a longitudinal axis 614 of an opening 612 and a longitudinal axis of an IV set. The protrusions 620a-c are oriented for attachment on one longitudinal end 650, and the protrusions 620d-f are oriented for attachment on another longitudinal end 652. This configuration may omit any protrusions that are oriented perpendicular to the longitudinal axis 614 of the opening 612 and a longitudinal axis of an IV set.

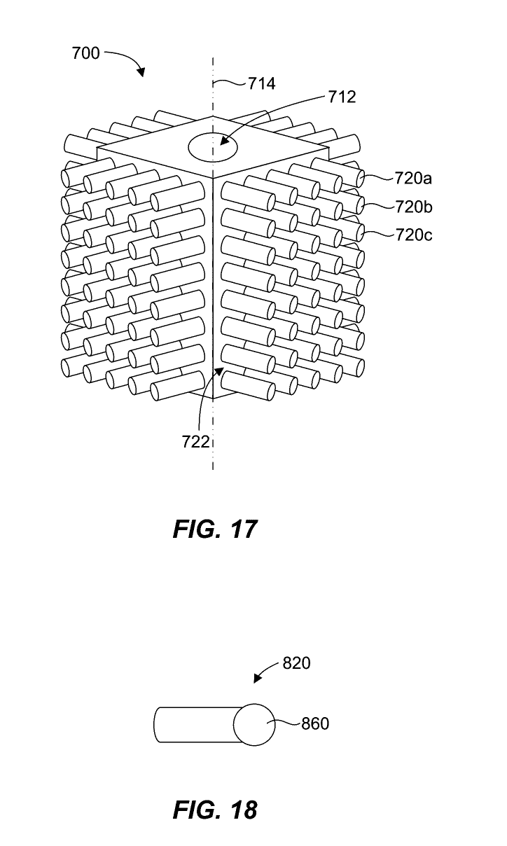

FIG. 17 illustrates an IV set management device 700 in accordance with another example of the present disclosure. Similar to some previous IV set management devices discussed above, the IV set management device 600 has a protrusion outer boundary (not shown for clarity) that is defined at least in part by a cuboid three-dimensional shape and defined at least in part by a plane (not shown for clarity). In addition, a projected outer boundary (not shown for clarity) of the protrusions in a direction parallel to a longitudinal axis 714 of an opening 712 is a rectangle. In this case, protrusions 720a-c of the IV set management device 700 are configured as rods, unlike the fins previously discussed. As with other examples disclosed herein, for simplicity, only three protrusions are specifically identified in the figure, although more protrusions are illustrated. Unlike the fin protrusions, the plurality of rod protrusions 720a-c can define only single recess 722 for a given “face” or “surface” of the IV set management device 700. Thus, for example, the rod protrusions 720a-c of the present example are present on four sides or faces of a cuboid. For each face, the rod protrusions are isolated or disconnected from one another and therefore a single continuous recess is formed between the rods. This aspect is discussed in more detail below with regard to the mating or coupling interfaces defined by the protrusions and the recess.

Although the IV set management device 700 depicted in FIG. 17 has a protrusion outer boundary that is defined at least in part by a cuboid three-dimensional shape, it should be recognized that an outer boundary of the rod protrusions can be defined by any suitable three-dimensional shape, such as at least in part by a cylinder as in examples discussed hereinabove. Similarly, the rod protrusions can be of any suitable three-dimensional shape or cross-section. For example, the rod protrusions can be configured as a cylinder (e.g., circular, elliptical, etc.), a prismatoid, a polytope (e.g., a uniform prismatic polytope), a cone (e.g., a spike), or any other suitable shape. In one aspect, the protrusions of an IV set management device can be a combination of fins and rods. Rod protrusions can have any suitable tip, such as planar, rounded, pointed, etc.

In one aspect, illustrated in FIG. 18, a rod protrusion 820 can have a tip configured as a coupling enhancement feature 860. A coupling enhancement feature can be configured to interface with protrusions and/or coupling enhancement features of a mating IV set management device. In this case, the coupling enhancement feature 860 comprises a spherical shape, although any suitable shape or geometry may be utilized.

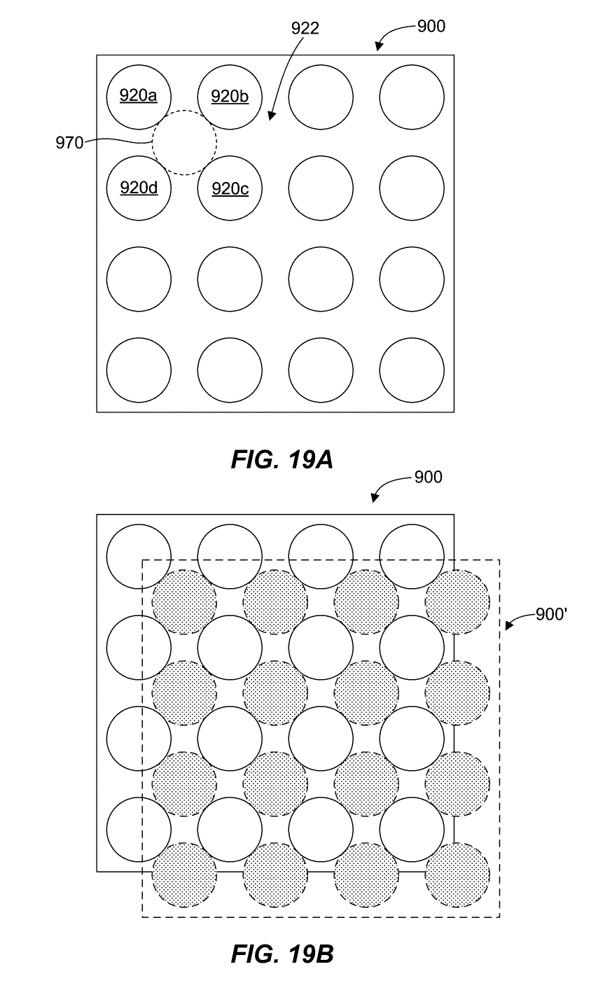

FIGS. 19A and 19B illustrate an example of how an IV set management device 900 having rod protrusions 920a-d can couple with another similarly configured IV set management device 900′. For simplicity, only four protrusions are specifically identified in the figure, although more protrusions are illustrated. In this example, the rod protrusions 920a-d have a circular cross section, which may be similar to the rod protrusions of FIG. 17. As shown in FIG. 19A, the rod protrusions 920a-d can define a recess 922. The rod protrusions 920a-d can also be sized and positioned to define an envelope boundary 970 within the recess 922 configured to mate with the protrusions 920a-d. Thus, the size of the envelope boundary 970 is substantially the same size as a cross-section of an individual rod protrusion 920a-d. In this case, a single envelope boundary 970 is defined by four rod protrusions 920a-d, each having a single point of contact in two dimensions (e.g., a tangential relationship in cross-section) with the envelope boundary 970 to fully constrain the envelope boundary 970 in two dimensions. In some embodiments, each rod protrusion can have a line contact with an envelope boundary in three dimensions. Multiple envelope boundaries can be defined by the rod protrusions 920a-d within the recess 922, and a single rod protrusion can define more than one envelope boundary. The rod protrusions 920a-d and the recess 922 can therefore define mating or coupling interfaces for coupling with a similarly configured IV set management device 900′, as shown in FIG. 19B. It should be recognized that the relative position of the IV set management devices 900, 900′ shown in FIG. 19B is only one possibility, as the IV set management devices 900, 900′ can be coupled to one another in a variety of different relative positions.

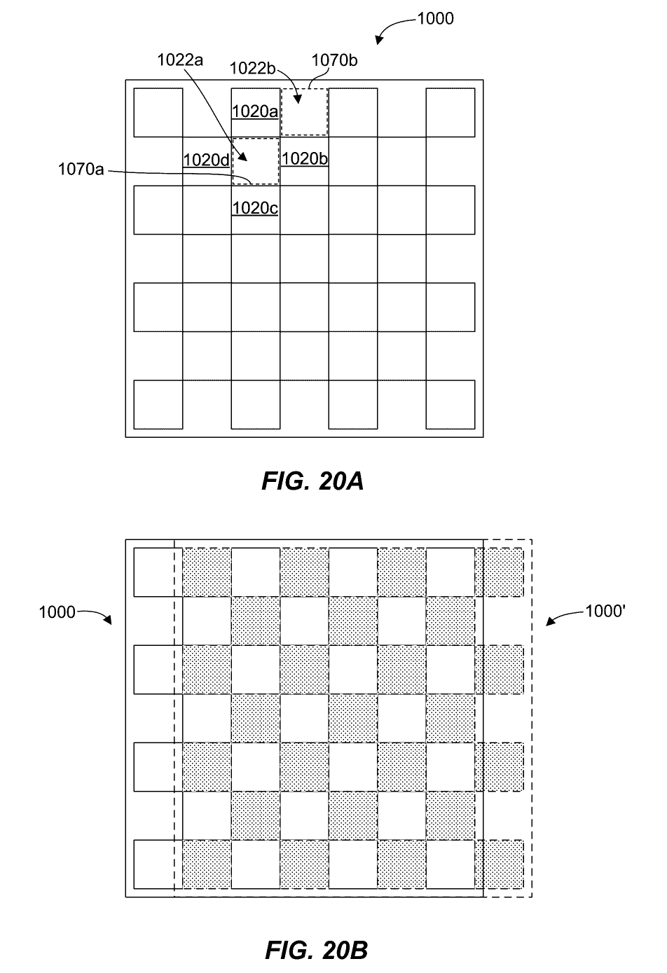

FIGS. 20A and 20B illustrate another example of how an IV set management device 1000 having rod protrusions 1020a-d can couple with another similarly configured IV set management device 1000′. For simplicity, only four protrusions are specifically identified in the figure, although more protrusions are illustrated. In this example, the rod protrusions 1020a-d have a rectangular or square cross-section. As illustrated, the rod protrusions 1020a-d are connected to an adjacent rod protrusion at one or more corners, although alternative rectangular or square cross-section rod protrusions can be physically isolated from one another. Therefore, as shown in FIG. 20A, the connected rod protrusions 1020a-d can define a plurality of recesses 1022a-b. For simplicity, only two recesses are specifically identified in the figures, although more recesses are illustrated. The rod protrusions 1020a-d can also be sized and positioned to define an envelope boundary 1070 within the recess 1022a configured to mate with the protrusions, which fully constrains the envelope boundary 1070 in two dimensions. In addition, rod protrusions can define an envelope boundary 1070b within the recess 1022b configured to mate with the protrusions, which constrains the envelope boundary 1070b in one dimension. This type of envelope boundary may be utilized and occupied by a mating protrusion, but cannot be relied on alone to fully constrain an IV set management device in two dimensions. The size of the envelope boundaries 1070a-b are substantially the same size as a cross-section of an individual rod protrusion 1020a-d, which may also be the same size as an individual recess 1022a. In this case, a single envelope boundary is defined by three or four rod protrusions, each having line contact in two dimensions with the envelope boundary. In some embodiments, each rod protrusion can have a plane contact with an envelope boundary in three dimensions. Multiple envelope boundaries 1070a-b can be defined by the rod protrusions 1020a-d in the plurality of recesses 1022a-b, and a single rod protrusion can define more than one envelope boundary. The rod protrusions 1020a-d and the recesses 1022a-b can therefore define mating or coupling interfaces for coupling with a similarly configured IV set management device 1000′, as shown in FIG. 20B. It should be recognized that the relative position of the IV set management devices 1000, 1000′ shown in FIG. 20B is only one possibility, as the IV set management devices 1000, 1000′ can be coupled to one another in a variety of different relative positions.

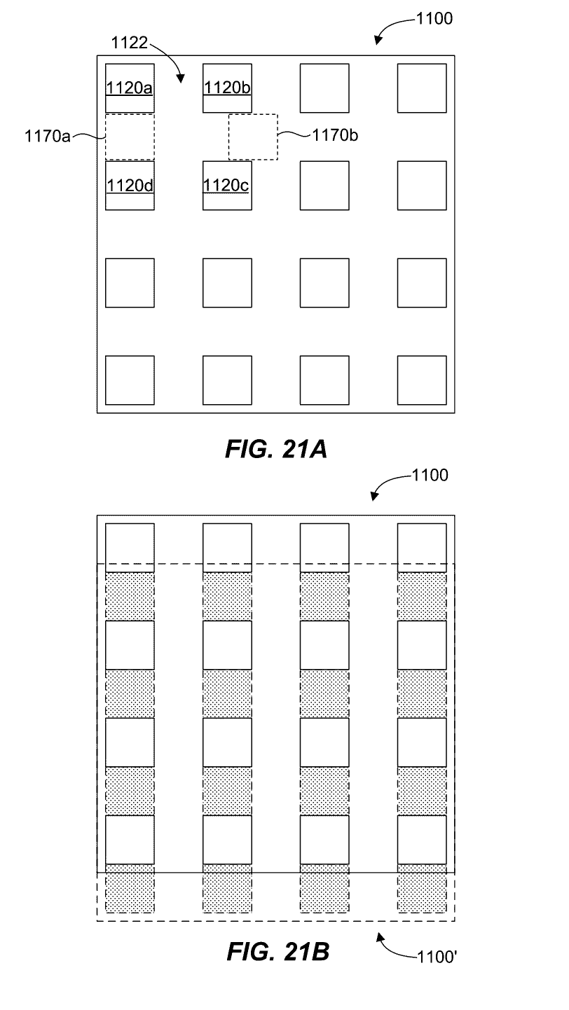

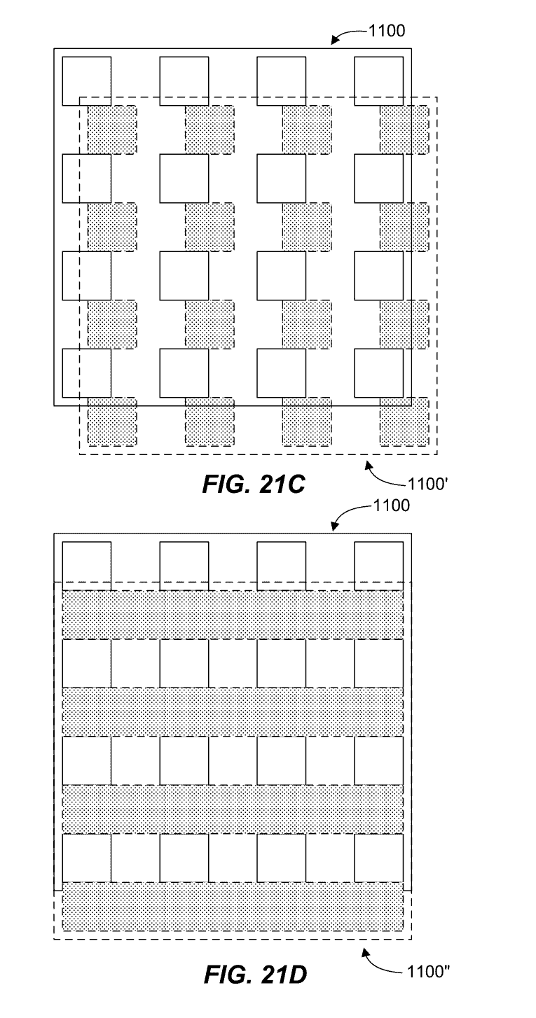

FIGS. 21A and 21B illustrate another example of how an IV set management device 1100 having rod protrusions 1120a-d can couple with another similarly configured IV set management device 1100′. In this example, the rod protrusions 1120a-d have a rectangular or square cross-section. As illustrated, the rod protrusions 1120a-d are physically isolated from one another. As shown in FIG. 21A, the rod protrusions 1120a-d can define a recess 1122. The rod protrusions 1120a-d can also be sized and positioned to define, at least in part, an envelope boundary 1170a within the recess 1122 configured to mate with the protrusions 1120a-d. Thus, the size of the envelope boundary 1170a is substantially the same size as a cross-section of an individual rod protrusion. In this case, a single envelope boundary 1170a is defined by two rod protrusions 1120a, 1120d, for example, each having line contact in two dimensions with the envelope boundary 1170a. In some embodiments, each rod protrusion can have a plane contact with an envelope boundary in three dimensions. Multiple envelope boundaries can be defined by the rod protrusions 1120a-d within the recess 1122, and a single rod protrusion can define more than one envelope boundary. The rod protrusions 1120a-d and the recess 1122 can therefore define mating or coupling interfaces for coupling with a similarly configured IV set management device 1100′, as shown in FIG. 21B. It should be recognized that the relative position of the IV set management devices 1100, 1100′ shown in FIG. 21B is only one possibility, as the IV set management devices 1100, 1100′ can be coupled to one another in a variety of different relative positions.

In this example, the configuration of the rod protrusions 1120a-d does not fully constrain the envelope boundary 1170a in two dimensions, as illustrated by envelope boundary 1170b, which is defined at least in part by rod protrusions 1120b, 1120c. The envelope boundary 1170b is shown in a shifted position from the rod protrusions 1120b, 1120c. The envelope boundary 1170b can therefore be constrained in one degree of freedom by the while being unconstrained in another degree of freedom, which can lead to a relative coupling position of IV set management devices 1100, 1100′ as illustrated in FIG. 21C.

Despite the lack of constraint in a degree of freedom, the rod protrusion configuration of FIG. 21A may be acceptable due to the flexibility provided in coupling with another IV set management device 1100″, as shown in FIG. 21D. In this case, the IV set management device 1100 having rod protrusions can also couple with an IV set management device 1100″ having fin protrusions. Therefore, a similarly configured mating or coupling interface for rod protrusions can include mating or coupling interfaces defined by rod or fin protrusions. Likewise, a similarly configured mating or coupling interface for fin protrusions can include mating or coupling interfaces defined by rod or fin protrusions. Thus, protrusion attributes such as shape, size, orientation, etc. can vary significantly among IV set management devices and still provide protrusions and recesses that define mating or coupling interfaces that are similarly configured. This example sheds light on how a single IV set management device can include both rod and fin protrusions.

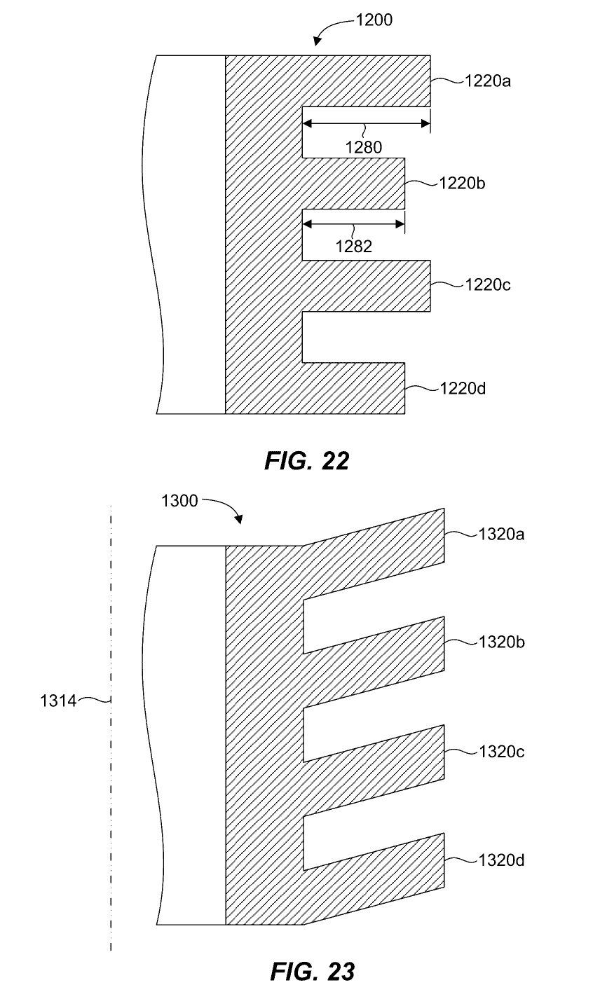

For example, as shown in FIG. 22, protrusions 1220a-d of an IV set management device 1200 can vary in length 1280, 1282 and can couple with another IV set management device having protrusions of different lengths or protrusions of the same length. In addition, an IV set management device 1300 can have protrusions 1320a-d that are not oriented perpendicular to a longitudinal axis 1314 of an opening for an IV set, as illustrated in FIG. 23. The IV set management device having such “angled” protrusions may couple with another “angled” IV set management device or with an IV set management device having perpendicularly oriented protrusions.

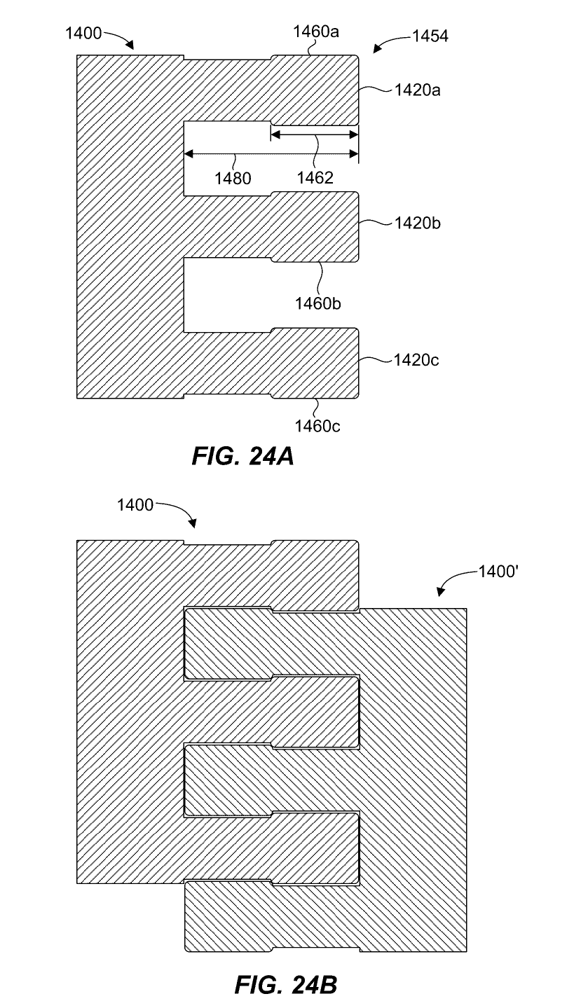

FIG. 24A illustrates a protrusion configuration of an IV set management device 1400 that includes coupling enhancement features in accordance with an example of the present disclosure. In this case, protrusions 1420a-c can have coupling enhancement features that include a locally expanded or widened portion 1460a-c toward a distal end 1454 of the protrusions. Such features can increase a coupling strength of the IV set management device 1400 with a similarly configured IV set management device 1400′, as shown in FIG. 24B. The locally expanded portions of the engaged protrusions can be configured to interface with one another to provide mechanical barriers to removing the protrusions from one another. In one aspect, a length 1462 of the locally expanded or widened portion toward the distal end 1454 can be about half of an overall length 1480 of the protrusions, although other configurations are possible. The increase in width for the locally expanded portion can vary as desired to allow the protrusions of one IV set management device to be received in recesses of another IV set management device 1400′. Material properties of the protrusions 1420a-c can be a factor in determining the exact dimensions.

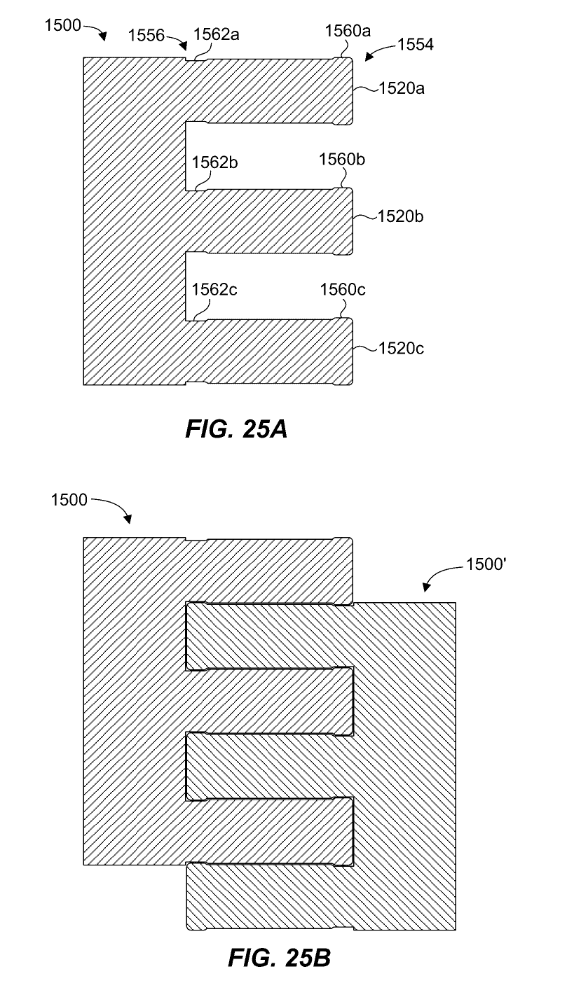

FIG. 25A illustrates a protrusion configuration of an IV set management device 1500 that includes coupling enhancement features in accordance with another example of the present disclosure. In this case, protrusions 1520a-c can have coupling enhancement features that include a locally expanded or widened portion 1560a-c toward a distal end 1554 of the protrusions, and a locally reduced or narrowed portion 1562a-c toward a proximal end 1556 of the protrusion. Such features can increase a coupling strength of the IV set management device 1500 with a similarly configured IV set management device 1500′, as shown in FIG. 25B. The locally expanded portions of the engaged protrusions can be configured to interface with the locally reduced portions to provide mechanical barriers to removing the protrusions from one another.

The cross-sections of protrusions shown in the figures can represent rod and/or fin protrusions. In addition, protrusion tips can be of any suitable configuration, such as flat, rounded, pointed, etc.

In accordance with one embodiment of the present invention, a method for facilitating management of an IV system is disclosed. The method can comprise providing an IV set management device having a body portion defining an opening to receive an IV set therein to couple the IV set management device to the IV set, a plurality of protrusions extending from the body portion, and at least one recess defined, at least in part, by at least one of the plurality of protrusions. The method can further comprise facilitating mating of the protrusions of the IV set management device with the at least one recess of the IV set management device to couple the IV set management device with a similarly configured second IV set management device. It is noted that no specific order is required in this method, though generally in one embodiment, these method steps can be carried out sequentially.

In one aspect of the method, the protrusions and the at least one recess of the IV set management device can define a mating interface, such that the IV set management device is configured to mate with the second IV set management device having a similarly configured mating interface. In another aspect of the method, the protrusions can be configured as fins, rods, or a combination thereof.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the foregoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

What is claimed is:

1. An IV set and IV set system management device, comprising:
a body portion defining an opening to receive an IV set therein to couple the IV set and IV set system management device to the IV set;
a plurality of protrusions extending from the body portion; and
at least one recess defined, at least in part, by at least one of the plurality of protrusions,
wherein the protrusions of the IV set and IV set system management device are configured to mate with the at least one recess of the IV set and IV set system management device, thus facilitating coupling of the IV set and IV set system management device with a similarly configured second IV set and IV set system management device.

2. The IV set and IV set system management device of claim 1, wherein the protrusions and the at least one recess of the IV set and IV set system management device define a mating interface, such that the IV set and IV set system management device is configured to mate with the second IV set and IV set system management device having a similarly configured mating interface.

3. The IV set and IV set system management device of claim 1, wherein the plurality of protrusions define an envelope boundary within the at least one recess configured to mate with the protrusions.

4. The IV set and IV set system management device of claim 1, wherein the at least one recess comprises a plurality of recesses.

5. The IV set and IV set system management device of claim 1, wherein the at least one recess comprises an inverted shape of the protrusions.

6. The IV set and IV set system management device of claim 1, wherein the body portion comprises a plurality of body portions coupleable to one another about the IV set to couple the IV set and IV set system management device to the IV set.

7. The IV set and IV set system management device of claim 1, wherein the body portion defines a lateral open end that provides access to the opening for the IV set.

8. The IV set and IV set system management device of claim 7, wherein the body portion extends greater than 180 degrees about a longitudinal axis of the opening to define the lateral open end, such that the IV set is laterally received in the opening and secured therein by the body portion.

9. The IV set and IV set system management device of claim 1, wherein the body portion defines a friction enhancing feature in the opening to engage the IV set and maintain the IV set and IV set system management device in a fixed position relative to the IV set during use.

10. The IV set and IV set system management device of claim 1, wherein an outer boundary of the plurality of protrusions is defined at least in part by a cylinder.

11. The IV set and IV set system management device of claim 1, wherein an outer boundary of the plurality of protrusions is defined at least in part by a plane.

12. The IV set and IV set system management device of claim 1, wherein a projected outer boundary of the protrusions in a direction parallel to a longitudinal axis of the opening comprises a circular shape, an elliptical shape, a rectangular shape, a triangular shape, a polygonal shape, a free-form shape, or a combination thereof.

13. The IV set and IV set system management device of claim 1, wherein the protrusions are configured as fins, rods, or a combination thereof.

14. The IV set and IV set system management device of claim 1, wherein at least some of the protrusions include coupling enhancement features to increase a coupling strength of the IV set and IV set system management device with the similarly configured second IV set and IV set system management device.

15. An IV set and IV set system management device, comprising:
a body portion defining an opening to receive an IV set therein to couple the IV set and IV set system management device to the IV set, the opening having a longitudinal axis;
a plurality of fins extending from the body portion; and
at least one recess defined, at least in part, by at least one of the plurality of fins,
wherein the plurality of fins are spaced apart from one another by the at least one recess along the longitudinal axis, and
wherein the protrusions of the IV set and IV set system management device are configured to mate with the at least one recess of the IV set and IV set system management device, thus facilitating coupling of the IV set and IV set system management device with a similarly configured second IV set and IV set system management device.

16. The IV set and IV set system management device of claim 15, wherein an outer boundary of the fins is defined at least in part by a cylinder.

17. The IV set and IV set system management device of claim 15, wherein an outer boundary of the fins is defined at least in part by a plane.

18. The IV set and IV set system management device of claim 15, wherein a gap of the at least one recess between protrusions is less than a diameter of the opening.

19. An IV set and IV set system management device, comprising:
a body portion defining an opening to receive an IV set therein to couple the IV set and IV set system management device to the IV set;
a plurality of rods extending from the body portion; and
at least one recess defined, at least in part, by at least one of the plurality of rods,
wherein the rods of the IV set and IV set system management device are configured to mate with the at least one recess of the IV set and IV set system management device, thus facilitating coupling of the IV set and IV set system management device with a similarly configured second IV set and IV set system management device.

20. The IV set and IV set system management device of claim 19, wherein an outer boundary of the rods is defined at least in part by a cylinder.

21. The IV set and IV set system management device of claim 19, wherein an outer boundary of the rods is defined at least in part by a plane.

22. AN IV set management system, comprising:
a first IV set and IV set system management device and a second IV set and IV set system management device, each IV set and IV set system management device having
a body portion defining an opening to receive an IV set therein to couple the IV set and IV set system management device to the IV set,
a plurality of protrusions extending from the body portion, and
at least one recess defined, at least in part, by at least one of the plurality of protrusions,
wherein the protrusions of the first IV set and IV set system management device are configured to mate with the at least one recess of the second IV set and IV set system management device, thus facilitating coupling of the first IV set and IV set system management device with the second IV set and IV set system management device.

23. The IV set management system of claim 22, wherein the opening of the first IV set and IV set system management device is configured to receive an IV set having a first diameter and the opening of the second IV set and IV set system management device is configured to receive an IV set having a second diameter.

24. The IV set management system of claim 22, further comprising a third IV set and IV set system management device, wherein the protrusions of the third IV set and IV set system management device are configured to mate with the at least one recess of the first and second IV set and IV set system management devices, thus facilitating coupling of the third IV set and IV set system management device with at least one of the first and second IV set and IV set system management devices.

25. The IV set management system of claim 22, wherein the first and second IV set and IV set system management devices have different numbers of protrusions.

26. The IV set management system of claim 22, wherein the protrusions of the first IV set and IV set system management device are configured as fins and the protrusions of the second IV set and IV set system management device are configured as rods.

27. A method for facilitating management of an IV system, comprising:
providing an IV set and IV set system management device having
a body portion defining an opening to receive an IV set therein to couple the IV set and IV set system management device to the IV set,
a plurality of protrusions extending from the body portion, and
at least one recess defined, at least in part, by at least one of the plurality of protrusions; and
facilitating mating of the protrusions of the IV set and IV set system management device with the at least one recess of the IV set and IV set system management device to couple the IV set and IV set system management device with a similarly configured second IV set and IV set system management device.

28. The method of claim 27, wherein the protrusions and the at least one recess of the IV set and IV set system management device define a mating interface, such that the IV set and IV set system management device is configured to mate with the second IV set and IV set system management device having a similarly configured mating interface.

29. The method of claim 27, wherein the protrusions are configured as fins, rods, or a combination thereof.
